(19) United States
(12) Patent Application Publication
(10) Pub. No.: US 2026/0102533 A1
(43) Pub. Date: Apr. 16, 2026

(54) TEAR-OPEN PERFUME DIFFUSION CONTAINER AND APPARATUS

(71) Applicant: FANER AROMA PRODUCT CO., LTD., Guangdong (CN)

(72) Inventors: Hsu-hui CHANG, Guangdong (CN); YongChang ZHANG, Guangdong (CN); Senlin YANG, Guangdong (CN); Songwen YE, Guangdong (CN)

(21) Appl. No.: 19/035,146

(22) Filed: Jan. 23, 2025

(30) Foreign Application Priority Data
Oct. 15, 2024 (CN) 202422492801.6

(51) Int. Cl. A61L 9/12; B65D 17/52

(52) U.S. Cl. CPC A61L 9/12; B65D 17/52; A61L 2209/131

(57) ABSTRACT

A tear-open perfume diffusion container and apparatus are disclosed. The diffusion container includes a container shell with at least one cavity, the at least one cavity having an opening, and at least one tear limiting platform is disposed in a same direction as the opening is convexly disposed at a bottom of the at least one cavity. A bottom of the at least one tear limiting platform is flexibly connected to the at least one cavity, such that the at least one tear limiting platform is movable relative to the container shell. A sealing tear sheet sealingly covers the opening, a bottom surface of the sealing tear sheet is provided with a fitting tear area that is connected and fixed to a top surface of the at least one tear limiting platform, and a diffusion film sealingly covers the opening and overlaps an outer side of the tear sheet.

### CROSS-REFERENCE TO RELATED APPLICATION

This application is based on and claims priority to Chinese Patent Application No. 202422492801.6, filed on October 15, 2024, the entirety of which is incorporated by reference herein.

### TECHNICAL FIELD

The present disclosure relates to the technical field of perfume diffusion, and in particular, to a tear-open perfume diffusion container and apparatus.

### BACKGROUND

Liquid perfume on the market is mainly packed in an airtight container. The opening of the airtight container is sealed by sequentially stacking a sealing sheet and a diffusion film. During use, the sealing sheet is broken to open the opening of the container, and the perfume in the container can evaporate to the outside through the diffusion film. At present, the container opening is opened typically by using a piercing tool for piercing through the sealing sheet or by applying an external pressure on the sealing sheet. Both of these methods are prone to damaging the diffusion film, causing the perfume to overflow.

### SUMMARY

An objective of the present disclosure is to provide a tear-open perfume diffusion container and apparatus, to solve one or more technical problems in the existing technologies, and at least provide a beneficial choice or create conditions.

The technical solution used to solve the above technical problem is as follows.

The present disclosure provides a tear-open perfume diffusion container, including:

a container shell provided with at least one cavity, the at least one cavity being provided with an opening, wherein at least one tear limiting platform that is disposed in a same direction as the opening is convexly disposed at a bottom of the at least one cavity, and a bottom of the at least one tear limiting platform is flexibly connected to the at least one cavity, such that the at least one tear limiting platform is movable relative to the container shell;

a sealing tear sheet sealingly covering the opening, wherein a bottom surface of the sealing tear sheet is provided with a fitting tear area that is connected and fixed to a top surface of the at least one tear limiting platform; and

a diffusion film sealingly covering the opening and overlapping an outer side of the sealing tear sheet.

The perfume diffusion container of the present disclosure has at least the beneficial effects.

In the present disclosure, the perfume in the cavity is sealed through the sealing tear sheet, when the perfume diffusion container needs to be opened for perfume evaporation and diffusion, the tear limiting platform is pressed or pulled to move relative to the container shell, to drive the fitting tear area to move, making the portion of the sealing tear sheet around the fitting tear area be torn to form a tear opening. The perfume evaporates outward through the diffusion film after passing through the tear opening of the sealing tear sheet. In the present disclosure, the fitting tear area on the sealing tear sheet is driven to move through the tear limiting platform, to achieve the tearing and opening of the sealing tear sheet, thus effectively reducing the risk of perfume overflow due to the damage of the diffusion film.

As a further improvement of the above technical solution, the sealing tear sheet is provided with a tear strip, and the tear strip is disposed around the fitting tear area.

As a further improvement of the above technical solution, the tear strip is in a thin sheet structure that is easily torn.

As a further improvement of the above technical solution, the tear strip is in a tear pattern structure.

As a further improvement of the above technical solution, an elastic connector is disposed between the bottom of the at least one tear limiting platform and the bottom of the at least one cavity, and the bottom surface of the sealing tear sheet and the top surface of the at least one tear limiting platform are in a same plane.

As a further improvement of the above technical solution, the container shell is provided with at least one diffusion step portion extending outward at an edge positioned at the opening of the container shell, the at least one cavity includes a main cavity and a diffusion cavity, the main cavity is communicated with the diffusion cavity through a plurality of circulation channels, the diffusion cavity is disposed in a middle of the diffusion step portion, the bottom surface of the sealing tear sheet is connected and fixed to a top surface of the diffusion step portion, and the at least one tear limiting platform is disposed in a middle of the diffusion cavity.

As a further improvement of the above technical solution, an inner step surface and an outer step surface that are disposed inside and outside are disposed on a periphery of the opening, the inner step surface and the outer step surface are spaced apart along a depth direction of the at least one cavity, an edge of the diffusion film is sealingly connected to the outer step surface, and an edge of the sealing tear sheet is sealingly connected to the inner step surface.

The present disclosure further provides a perfume diffusion apparatus including the perfume diffusion container as described above, a casing and an elastic pressing member. The perfume diffusion container is mounted in the casing, the elastic pressing member is provided with an actuating portion positioned corresponding to a top of the at least one tear limiting platform, and the actuating portion is configured to drive the at least one tear limiting platform to move in response to the elastic pressing member being pressed, so as to tear the sealing tear sheet.

As a further improvement of the above technical solution, the actuating portion is disposed on a top side of the container shell, and the actuating portion abuts against the top of the at least one tear limiting platform through the diffusion film and the sealing tear sheet.

As a further improvement of the above technical solution, the actuating portion is disposed on a bottom side of the container shell, the bottom of the at least one tear limiting platform is provided with a tear slot recessed inward from the bottom side of the container shell, the tear slot is provided with a clamping portion, and the actuating portion is clamped with the clamping portion.

Additional features and advantages of the present disclosure will be set forth in the description which follows, and in part will be apparent from the description, or may be learned by practice of the present disclosure.

### BRIEF DESCRIPTION OF DRAWINGS

The present disclosure will be further described below in conjunction with the accompanying drawings and embodiments.

FIG. 1 is an exploded view of a perfume diffusion container provided by an embodiment of the present disclosure;

FIG. 2 is a front cross-sectional view of a perfume diffusion container provided by an embodiment of the present disclosure;

FIG. 3 is an exploded view of a perfume diffusion apparatus provided by Embodiment I of the present disclosure;

FIG. 4 is a front cross-sectional view of a perfume diffusion apparatus provided by Embodiment I of the present disclosure;

FIG. 5 is an exploded view of a perfume diffusion apparatus provided by Embodiment II of the present disclosure; and

FIG. 6 is a front cross-sectional view of a perfume diffusion apparatus provided by Embodiment II of the present disclosure.

### Reference numerals:

perfume diffusion container 100;

container shell 200; cavity 210; opening 211; inner step surface 212; outer step surface 213; tear limiting platform 214; tear slot 215; clamping portion 216; elastic connector 217; diffusion step portion 218; main cavity 220; diffusion cavity 230; circulation channel 240; placing bottom surface 250; hollow area 260;

sealing tear sheet 300; fitting tear area 310; tear strip 320;

diffusion film 400;

casing 500;

actuating portion 600;

the first elastic pressing member 700;

the second elastic pressing member 800.

### DETAILED DESCRIPTION

The embodiments of the present disclosure are described in detail in this section, some embodiments of the present disclosure are presented in the drawings, which are configured to supplement the description of the written part of the specification by figures, so that people can directly and vividly understand each technical feature and the whole technical solution of the present disclosure, but cannot be understood as limitations to the present disclosure.

In the description of the present disclosure, it is understood that orientation or position relationships indicated by terms such as “upper”, “lower”, “front”, “rear”, “left”, and “right”, and the like are based on the orientation or position relationships as shown in the drawings, which are provided for ease of describing the present disclosure and simplifying the description only, rather than indicating or implying that the mentioned apparatus or element necessarily has a particular orientation and must be constructed and operated in the particular orientation. Therefore, these terms should not be understood as limitations to the present disclosure.

In the description of the present disclosure, the meaning of “several” is one or more, the meaning of “a plurality of” is two or more, terms such as “greater than”, “less than”, “exceeding”, and the like are understood as excluding the specified number, and terms such as “above”, “below”, “within”, and the like are understood as including the specified number.

In the description of the present disclosure, unless specifically defined otherwise, terms such as “set”, “install”, “join”, and the like should be generally understood. Those of ordinary skill in the art may reasonably determine the specific meaning of the terms in the present disclosure in combination with the specific contents of the technical solution.

Liquid perfume on the market is mainly packed in an airtight container. The opening of the airtight container is sealed by sequentially stacking a sealing tear sheet and a diffusion film. During use, the sealing tear sheet is broken to open the opening of the container, and the perfume in the container can evaporate to the outside through the diffusion film. At present, the container opening is opened typically by using a piercing tool for piercing through the sealing sheet or by applying an external pressure on the sealing sheet. Both of these methods are prone to damaging the diffusion film, causing the perfume to overflow. In order to address the above problem, the present disclosure proposes a tear-open perfume diffusion container, to avoid damaging the diffusion film and causing the perfume to overflow when tearing the sealing tear sheet.

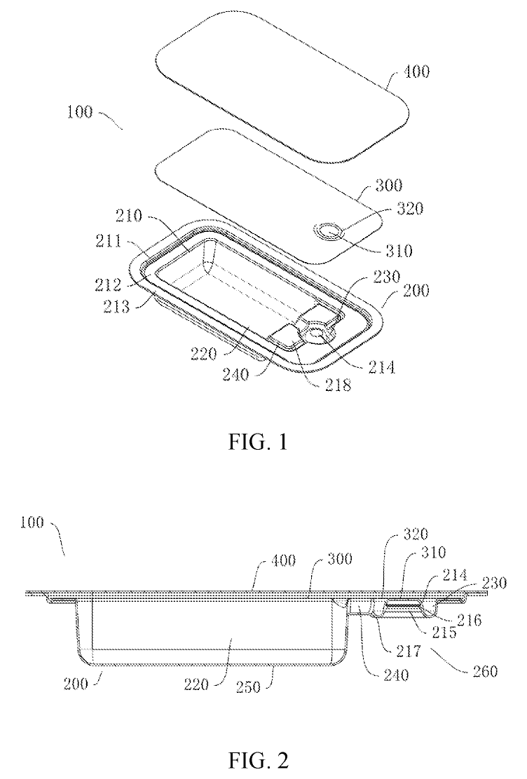

As shown in FIGS. 1 and 2, the perfume diffusion container 100 according to an embodiment of the present disclosure includes a container shell 200, a sealing tear sheet 300 and a diffusion film 400.

The container shell 200 in this embodiment is provided with a cavity 210 for holding perfume, and the cavity 210 is provided with an opening 211 facing upward. In other embodiments, the opening 211 of the cavity 210 can face downward, left, right, forward or backward. To facilitate the description of other components, this embodiment takes the opening 211 of the cavity 210 facing upward as an example for further description. In actual use, the container shell 200 is placed upright, with the opening 211 of the cavity 210 facing sideways.

The container shell 200 in this embodiment is produced using vacuum forming, the container shell 200 is in a box shape and has a certain elastic deformation function. In other embodiments, the container shell 200 may be made of other materials, and may be formed through methods such as stamping, injection molding, thermoforming, without further elaboration here.

The sealing tear sheet 300 in this embodiment is used to block and seal the opening 211, the sealing tear sheet 300 is made of a material that is easily torn. Specifically, in this embodiment, the sealing tear sheet 300 is made of an aluminum foil that is easily torn, the tearing sheet 300 is sealingly disposed at the opening 211, the sealing tear sheet 300 is sealingly connected to the surrounding edges of the opening 211, the sealing tear sheet 300 and the cavity 210 form an accommodating cavity, and the accommodating cavity is used for storing perfume.

The diffusion film 400 is used for the evaporation and diffusion of perfume, and the diffusion film 400 can be waterproof and breathable. The diffusion film 400 in this embodiment adopts a waterproof and breathable film, and the diffusion film 400 is a film structure with a certain elastic deformation. In some other embodiments, the diffusion film 400 may adopt other waterproof and breathable film structures.

The diffusion film 400 in this embodiment is also sealingly disposed at the opening 211, the diffusion film 400 is sealingly connected to the edges of the opening 211, the sealing tear sheet 300 and the diffusion film 400 are disposed inside and outside, and the diffusion film 400 in this embodiment overlaps outside the sealing tear sheet 300.

In this embodiment, an inner step surface 212 and an outer step surface 213 that are disposed inside and outside are disposed on a periphery of the opening 211, the inner step surface 212 and the outer step surface 213 are spaced apart along a depth direction of the cavity 210, an edge of the diffusion film 400 is sealingly connected to the outer step surface 213, and an edge of the sealing tear sheet 300 is sealingly connected to the inner step surface 212.

In this embodiment, the inner step surface 212 and the outer step surface 213 can facilitate the sealing connection for the diffusion film 400 and the sealing tear sheet 300, and can also form a gap between the diffusion film 400 and the sealing tear sheet 300. When pressing the outside diffusion film 400, the sealing tear sheet 300 will not be pressed directly. Only after pressing the diffusion film 400 to a certain extent, will it come into contact with the sealing tear sheet 300. At this time, the sealing tear sheet 300 can be pressed when the diffusion film 400 continues to be pressed, thus achieving the effect of preventing accidental contact. The gap is determined through a height difference between the inner step surface 212 and the outer step surface 213.

During production, the perfume is added into the cavity 210 in advance, then the opening 211 is sealed through the sealing tear sheet 300, and then the diffusion film 400 is placed over the opening 211 to form a diffusion container filled with the perfume.

In this embodiment, at least one tear limiting platform 214 is convexly disposed at a bottom of the cavity 210, and the tear limiting platform 214 is disposed in a same direction as the opening 211. In some embodiments, a plurality of tear limiting platforms 214 may be provided according to the requirements of the diffusion amount, and one tear limiting platform 214 is provided in this embodiment.

A bottom of the tear limiting platform 214 in this embodiment is flexibly connected to the bottom of the cavity 210, so that the tear limiting platform 214 can move relative to the container shell 200. It can be understood that under the action of an external force, the top of the tear limiting platform 214 can move relative to the container shell 200, and the top of the tear limiting platform 214 can move in the depth direction or swing left and right. The tear limiting platform 214 is deflected under the action of the external force, causing the sealing tear sheet 300 to be torn open. In this embodiment, the top of the tear limiting platform 214 is defined to move towards the bottom of the container shell 200.

A bottom surface of the sealing tear sheet 300 is provided with at least one fitting tear area 310 which is positioned corresponding to the tear limiting platform 214, and a top surface of the tear limiting platform 214 is connected and fixed to the fitting tear area 310. In some embodiments, the top surface of the tear limiting platform 214 and the fitting tear area 310 may be adhered by glue to achieve fixation, or fixed through laser welding or heat sealing.

Moreover, the bottom surface of the sealing tear sheet 300 and the top surface of the tear limiting platform 214 are in a same plane. In a natural state, the fitting tear area 310 is coplanar with other parts of the sealing tear sheet 300.

When the perfume diffusion container 100 needs to be opened for perfume diffusion, under the action of the external force, the tear limiting platform 214 is pressed or pulled to move relative to the container shell 200, to drive the fitting tear area 310 to move. This motion causes the surrounding sealing tear sheet 300 to tear open to form a tear opening. The perfume evaporates outward through the diffusion film 400 after passing through the tear opening of the sealing tear sheet 300. In the present disclosure, the fitting tear area 310 on the sealing tear sheet 300 is driven to move through the tear limiting platform 214, to achieve the tearing and opening of the sealing tear sheet 300, thus effectively reducing the risk of perfume overflow due to damaging the diffusion film 400.

To further avoid the damage to the diffusion film 400, in this embodiment, the tear limiting platform 214 can be pressed or pulled downward along the depth direction, so that the fitting tear area 310 moves downward. When the external force acts on the tear limiting platform 214, the stress is concentrated in the fitting tear area 310. At this time, due to the fact that the periphery of the sealing tear sheet 300 is sealingly connected to the edges of the opening 211, the tear opening is formed at the fitting tear area 310.

In addition, the tear limiting platform 214 in this embodiment not only plays a role in tearing the fitting tear area 310, but also limits the tearing movement of the fitting tear area 310. When the external force acts on the top of the tear limiting platform 214 from top to bottom through the diffusion film 400 and the sealing tear sheet 300, i.e., the tear limiting platform 214 is pressed down, the downward movement of the fitting tear area 310 can also be achieved. In this case, the support of the tear limiting platform 214 plays a role in limiting the pressing stroke. It can be understood that the tear limiting platform 214 cannot be pressed down infinitely. If the tear limiting platform 214 is pressed down too deep, the diffusion film 400 may be pressed down to a critical point where the diffusion film 400 is broken.

The number of the cavity 210 may one or more. When a plurality of cavities 210 are provided, each cavity 210 can store perfumes of different perfumes. During use, the tear limiting platforms 214 of the different cavities 210 can be pressed or pulled, to achieve the mixing of different perfumes.

To make the sealing tear sheet 300 be easier to tear, the sealing tear sheet 300 in this embodiment is provided with a tear strip 320 that is disposed around the fitting tear area 310 and formed by laser etching a polyethylene (PE) film. The tear strip 320 is easier to tear than the area of the sealing tear sheet 300. When the sealing tear sheet 300 is pulled, it will tear along the tear strip 320 to form the tear opening.

The tear strip 320 is in a thin sheet structure that is easily torn. In some embodiments, the tear strip 320 is in a tear pattern structure created by laser or similar techniques to guide the tearing action.

When the pulling way is used to drive the tear limiting platform 214 to move down in the depth direction, in order to facilitate the application of the external force, the bottom of the tear limiting platform 214 in this embodiment is provided with a tear slot 215 recessed inward from the bottom of the container shell 200, and the tear slot 215 is provided with a clamping portion 216. When in use, the external pulling component extends into the tear slot 215 and clamps with the clamping portion 216 to achieve the connection between the external pulling component and the top of the tear limiting platform 214, and at this time the tear limiting platform 214 can be pulled downward.

In this embodiment, the tear limiting platform 214 and the cavity 210 are integrally formed. It can be understood that the tear limiting platform 214 and the container shell 200 are integrally formed by vacuum forming of polyethylene terephthalate (PET). When the container shell 200 is produced, the tear limiting platform 214 and the cavity 210 can be integrally formed, to simplify the production process.

In this embodiment, an elastic connector 217 is disposed between the bottom of the tear limiting platform 214 and the bottom of the cavity 210, and the free movement of the tear limiting platform 214 under the action of the external force is achieved through the elastic deformation of the elastic connector 217.

According to the molding method, the elastic connector 217 in this embodiment is annular, and a cross section of the elastic connector 217 is arc-shaped. The free movement of the tear limiting platform 214 along the depth direction is achieved through the elastic connector 217, and the elastic connector 217 also has elasticity for easy operation. The movement limiting of the tear limiting platform 214 is also achieved through the elastic deformation of the elastic connector 217.

Moreover, the tear limiting platform 214 has a tapered outer diameter from bottom to top along the depth direction, which can improve the structural strength of the tear limiting platform 214, prevent the tear limiting platform 214 from tilting, and also facilitate mold release during injection molding.

Furthermore, the container shell 200 is provided with at least one diffusion step portion 218 extending outward at an edge of the opening 211, the cavity 210 includes a main cavity 220 and a diffusion cavity 230, the main cavity 220 is communicated with the diffusion cavity 230 through a plurality of circulation channels 240, the diffusion cavity 230 is disposed in the middle of the diffusion step portion 218, the bottom surface of the sealing tear sheet 300 is connected and fixed to a top surface of the diffusion step portion 218, and the tear limiting platform 214 is disposed in the middle of the diffusion cavity 230. It can be understood that the top surface of the diffusion step portion 218 and the inner step surface 212 are on a same plane, so that the portions of the sealing tear sheet 300 around the diffusion cavity 230 are all fixed. When the tear limiting platform 214 moves, the tear stress acts around the fitting tear area 310, making the sealing tear sheet 300 be easier to be torn.

In this embodiment, the main cavity 220 is mainly used to hold and store perfume, while the diffusion cavity 230 is mainly used for the evaporation of perfume, the tear limiting platform 214 provided in the diffusion cavity 230 corresponds to the fitting tear area 310 on the sealing tear sheet 300. Over time, the perfume in the main cavity 220 will evaporate outward along the circulation channels 240, the diffusion cavity 230, and the diffusion film 400.

As shown in FIG. 2, a bottom of the container shell 200 facing away from the opening 211 is provided with a placing bottom surface 250, a hollow area 260 is formed between the bottom of the diffusion step portion 218 and the plane where the placing bottom surface 250 is located, such that a height difference is formed between the diffusion cavity 230 and the main cavity 220. This provides a passage for the perfume to evaporate or pass through, and also facilitates the formation of the tear limiting platform 214 and provides space for mounting external pulling component.

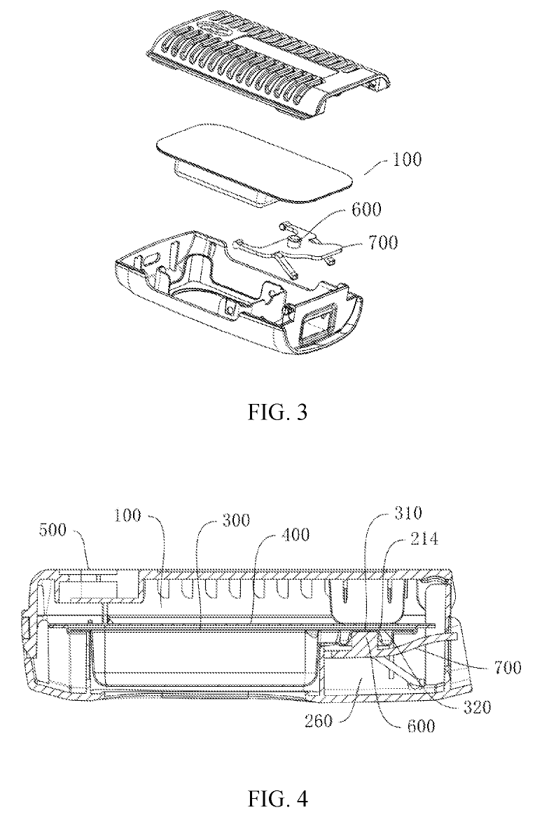

The present disclosure further provides a perfume diffusion apparatus. As shown in FIGS. 3 to 6, the perfume diffusion apparatus includes the above-mentioned perfume diffusion container 100, a casing 500 and an elastic pressing member. The perfume diffusion container 100 is mounted in the casing 500, the elastic pressing member is provided with an actuating portion 600 which is positioned corresponding to a top of the tear limiting platform 214, and the actuating portion 600 is configured to drive the tear limiting platform 214 to move when the elastic pressing member is pressed, so as to tear the sealing tear sheet 300.

The elastic pressing member is provided with a connecting end and an operating end, the connecting end is connected to an interior of the casing 500 while the operating end can swing freely, and the actuating portion 600 is disposed between the connecting end and the operating end. When in use, the elastic pressing member is driven to swing by pressing the operating end, so as to drive the actuating portion 600 to move, and under the action of the actuating portion 600, the tear limiting platform 214 moves in the depth direction to tear the sealing tear sheet 300.

Regarding the position of the actuating portion 600, the present disclosure proposes two embodiments:

As shown in FIGS. 3 and 4, which is Embodiment I:

In this embodiment, a first elastic pressing member 700 positioned at a bottom side of the perfume diffusion container 100 is disposed in the casing 500, the actuating portion 600 is disposed at a bottom side of the container shell 200, and the actuating portion 600 is connected to the tear limiting platform 214 through the bottom surface of the container shell 200. It can be understood that in this case, the actuating portion 600 extends into the tear slot 215 and is clamped with the clamping portion 216, to achieve the connection between the actuating portion 600 and the top of the tear limiting platform 214. When the operating end is pressed down, the tear limiting platform 214 is pulled downward through the actuating portion 600.

In some other embodiments, the actuating portion 600 can also act on the tear limiting platform 214 through adhesives, suction cups, or other methods.

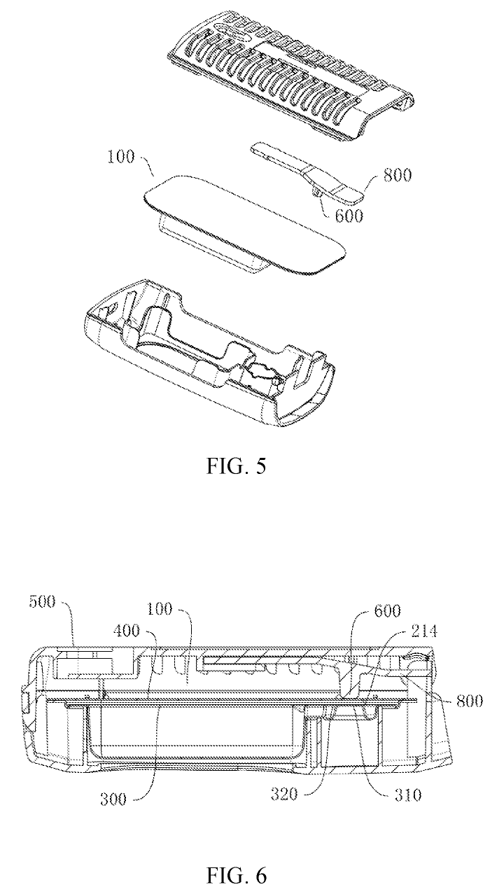

As shown in FIGS. 5 and 6, which is Embodiment II:

In this embodiment, a second elastic pressing member 800 positioned at a top side of the perfume diffusion container 100 is disposed in the casing 500, the actuating portion 600 is disposed at the top side of the container shell 200, and the actuating portion 600 abuts against the top of the tear limiting platform 214 through the diffusion film 400 and the sealing tear sheet 300. It can be understood that in this case, the actuating portion 600 is positioned on the upper side of the diffusion film 400, when the operating end is pressed downward, the top of the tear limiting platform 214 is pressed down through the actuating portion 600. In this case, the pressing stroke of the tear limiting platform 214 is less than the critical deformation stroke at which the diffusion film 400 is destroyed.

Some embodiments of the present disclosure are specifically described above, but the present disclosure is not limited to the above embodiments. Those having ordinary skills in the art can also make various equivalent modifications or substitutions without departing from the gist of the present disclosure, and these equivalent modifications or substitutions are included in the scope defined by the claims of the present disclosure.

What is claimed is:

1. A tear-open perfume diffusion container, comprising:
a container shell provided with at least one cavity, the at least one cavity being provided with an opening, wherein at least one tear limiting platform that is disposed in a same direction as the opening is convexly disposed at a bottom of the at least one cavity, and a bottom of the at least one tear limiting platform is flexibly connected to the at least one cavity, such that the at least one tear limiting platform is movable relative to the container shell;
a sealing tear sheet sealingly covering the opening, wherein a bottom surface of the sealing tear sheet is provided with a fitting tear area that is connected and fixed to a top surface of the at least one tear limiting platform; and
a diffusion film sealingly covering the opening and overlapping an outer side of the sealing tear sheet.

2. The perfume diffusion container of claim 1, wherein the sealing tear sheet is provided with a tear strip, and the tear strip is disposed around the fitting tear area.

3. The perfume diffusion container of claim 2, wherein the tear strip is in a thin sheet structure that is easily torn.

4. The perfume diffusion container of claim 2, wherein the tear strip is in a tear pattern structure.

5. The perfume diffusion container of claim 1, wherein an elastic connector is disposed between the bottom of the at least one tear limiting platform and the bottom of the at least one cavity, and the bottom surface of the sealing tear sheet and the top surface of the at least one tear limiting platform are in a same plane.

6. The perfume diffusion container of claim 1, wherein the container shell is provided with at least one diffusion step portion extending outward at an edge positioned at the opening of the container shell, the at least one cavity comprises a main cavity and a diffusion cavity, the main cavity is communicated with the diffusion cavity through a plurality of circulation channels, the diffusion cavity is disposed in a middle of the diffusion step portion, the bottom surface of the sealing tear sheet is connected and fixed to a top surface of the diffusion step portion, and the at least one tear limiting platform is disposed in a middle of the diffusion cavity.

7. The perfume diffusion container of claim 1, wherein an inner step surface and an outer step surface that are disposed inside and outside are disposed on a periphery of the opening, the inner step surface and the outer step surface are spaced apart along a depth direction of the at least one cavity, an edge of the diffusion film is sealingly connected to the outer step surface, and an edge of the sealing tear sheet is sealingly connected to the inner step surface.

8. A perfume diffusion apparatus comprising the perfume diffusion container of claim 1, a casing and an elastic pressing member, wherein the perfume diffusion container is mounted in the casing, the elastic pressing member is provided with an actuating portion positioned corresponding to a top of the at least one tear limiting platform, and the actuating portion is configured to drive the at least one tear limiting platform to move in response to the elastic pressing member being pressed, so as to tear the sealing tear sheet.

9. The perfume diffusion apparatus of claim 8, wherein the actuating portion is disposed on a top side of the container shell, and the actuating portion abuts against the top of the at least one tear limiting platform through the diffusion film and the sealing tear sheet.

10. The perfume diffusion apparatus of claim 8, wherein the actuating portion is disposed on a bottom side of the container shell, the bottom of the at least one tear limiting platform is provided with a tear slot recessed inward from the bottom side of the container shell, the tear slot is provided with a clamping portion, and the actuating portion is clamped with the clamping portion.
